(19) United States
(12) Patent Application Publication
(10) Pub. No.: US 2003/0185774 A1
(43) Pub. Date: Oct. 2, 2003

(54) Cosmetic coating composition comprising carboxyalkyl cellulose ester

(76) Inventors: Suzanne Winegar Dobbs, Kingsport, TN (US); Terry Ann Oldfield, Kingsport, TN (US); Jessica Dee Posey-Dowty, Kingsport, TN (US)

Correspondence Address: Jonathan D. Wood, Eastman Chemical Company, P.O. Box 511, Kingsport, TN, 37662-5075, US

(21) Appl. No.: 10/114,353

(22) Filed: Apr. 2, 2002

(51) Int. Cl. A61K 7/04

(52) U.S. Cl. 424/61

(57) ABSTRACT

Disclosed are cosmetic film-forming compositions which are especially useful as nail coating compositions which, upon drying, form a film which will adhere to the keratin of the nail, i.e., fingernails and toenails, and to artificial nails. More specifically, the present invention pertains to cosmetic coating compositions comprising one or more carboxyalkyl cellulose (CAC) esters and a liquid vehicle or solvent. The compositions may be anhydrous or contain up to about 80% water. While primarily useful as nail coatings, the coating compositions also may be applied to the skin to provide a pigmented coating or glossy finish.

### FIELD OF THE INVENTION

This invention pertains to cosmetic film-forming compositions which are especially useful as nail coating compositions which, upon drying, form a film which will adhere to the keratin of the nail, i.e., fingernails and toenails; and to artificial nails. More specifically, the present invention pertains to cosmetic film-forming compositions comprising one or more carboxyalkyl cellulose (CAC) esters and a liquid vehicle or solvent.

### BACKGROUND OF THE INVENTION

Currently-available, commercial nail coating compositions typically contain at least 50% to 70% C₁-C₅ alkyl acetate solvents, which have a strong odor. The alkyl acetate solvents often are used in combination with a smaller amount of a C₂-C₅ alcohol. The primary polymer used in these products as the film-former or binder is nitrocellulose, often in combination with a co-resin. Cellulose acetate propionate and cellulose acetate butyrate also are used in nail enamels as the primary or secondary film-former with solvents such as ethyl acetate and butyl acetate.

Nail coating compositions which utilize organic solvents are well-known in the art. U.S. Pat. No. 5,785,958 describes the use of a combination of cellulose acetate butyrate, a methacrylate monomer, methacrylate polymer, cross-linking agent, and initiator. The solvent system is a combination of ethyl acetate, butyl acetate, and isopropyl alcohol. U.S. Pat. No. 5,747,019 describes a clear nail polish top coat comprising a cellulose acetate propionate ester dissolved in a mixture of solvents free of toluene. The solvent mixture consists of ethyl acetate, ethyl alcohol, isobutyl acetate, and methyl ethyl ketone. Cellulose acetate propionate and cellulose acetate butyrate are typically used in nail polish top coats rather than in base coats, because their adhesion to the nail is deficient compared to nitrocellulose. U.S. Pat. Nos. 5,720,804 and 5,512,273 describe a nail top coat composition comprising at least one cellulose ester in a solvent system consisting of an aliphatic solvent and a cycloaliphatic solvent. The aliphatic solvent is further described as consisting of at least one aliphatic ester.

U.S. Pat. No. 5,424,061 describes a quick-drying nail enamel composition achieved through the use of 4.5-35% acetone in the solvent system. U.S. Pat. No. 5,346,692 discloses a nail lacquer for treating onychomycosis. The solvent system comprises 50-70% acetone and 30-50% ethanol, wherein the polymeric film-forming agent is first dissolved in acetone. U.S. Pat. No. 5,130,125 describes a clear top coat composition consisting of cellulose acetate butyrate ester dissolved in a mixture of solvents consisting of toluene and n-butyl acetate. U.S. Pat. No. 4,712,571 describes a low viscosity nail polish composition comprising about 2.0-40% of a film former selected from nitrocellulose, cellulose propionate, cellulose acetate butyrate, ethyl cellulose, sucrose acetate isobutyrate, and others.

Many of the water-based nail coating compositions use vinyl polymers or a combination of vinyl polymers and condensation polymers as the film former. Examples of water-based compositions include the water-based fingernail coating composition containing 40-70% water, 5-20% C₁-C₄ alcohol, and an emulsion polymer described as an acrylic copolymer, styrene-acrylic copolymer, urethane-acrylic copolymer, or mixtures thereof as described in U.S. Pat. No. 5,955,063. U.S. Pat. No. 5,716,603 describes aqueous nail polish compositions comprising an acrylic resin crosslinked with acrylated urethane oligomers. U.S. Pat. Nos. 5,266,322 and 5,380,520 describe a water-based cosmetic film forming composition comprising a sulfopolyester, a copolymer based on vinyl acetate and dialkyl maleate or fumarate, and the reaction product of acetoacetoxyethyl alkylacrylate with a vinyl-functional monomer.

U.S. Pat. No. 5,120,529 describes a water-based nail polish comprising a polyurethane and/or a polyurethane copolymer as binder, in combination with an acrylate-styrene copolymer. Cellulose and cellulose derivatives are listed as possible thickeners for this system. U.S. Pat. No. 4,158,053 describes aqueous emulsion polymer nail coating formulations. The polymers described are prepared from vinyl monomers. Donald Brody, Water-Based Fingernail Coating, Surface Coatings Australia, November 1998, describes acrylic and styrene-acrylic copolymers as binders (film-formers) for water-based fingernail enamel. Water-soluble cellulosic resins are referred to as conventional thickeners for water-based systems. No mention is made of cellulosic resins being useful as film-formers.

U.S. Pat. Nos. 5,741,901; 5,871,573; and 5,981,738 describe modified cellulose esters containing unsaturated pendent groups that are curable upon exposure to ultraviolent (UV) radiation. The cellulose ester is described as useful as a nail polish. Also described is a waterborne coating composition comprising the modified cellulose ester, where at least 25% of the carboxyl groups of the modified cellulose ester are neutralized with an amine or ammonia. The coating composition also includes 5% -30% water-miscible solvent and 40% -70% water.

U.S. Pat. Nos. 5,668,273 and 5,792,856 describe certain C₂-C₄ alkanoate esters carboxy(C₁-C₃ alkyl) cellulose, processes for the preparation thereof, and coating compositions, including waterborne coating compositions, containing one of the disclosed carboxy(C₁-C₃ alkyl) cellulose esters. U.S. Pat. No. 5,994,530 describes aqueous pigment dispersions comprising a pigment and a C₂-C₄ alkanoate esters carboxy(C₁-C₃ alkyl) cellulose.

### BRIEF SUMMARY OF THE INVENTION

We have found that C₂-C₄ alkanoate esters carboxy(C₁-C₃ alkyl) cellulose are useful film-formers when used in cosmetic coating compositions such as coating compositions designed for application to finger and toe nails. Thus, one embodiment of the present invention pertains to a cosmetic coating composition comprising:

(1) about 1 to 40 weight percent, based on the total weight of (1) and (2), of an unneutralized or partially neutralized carboxyalkyl cellulose ester; and

(2) about 60 to 99 weight percent, based on the total weight of (1) and (2), of a liquid vehicle comprising

(i) 0 to about 80 weight percent water;

(ii) about 2 to 25 weight percent of an organic solvent that evaporates slower than water selected from ether-alkanols, carboxylate esters of ether-alkanols, mono-carboxylate esters of diols containing from about 6 to 16 carbon atoms, plasticizers, and mixtures of any two or more thereof; and

(iii) 0 to about 88 weight percent of a fast-evaporating solvent selected from one or more aliphatic, straight- or branched-chain alkanols having 2 to 4 carbon atoms; aliphatic, straight-chain ketones having 3 to 5 carbon atoms; and lower alkyl carboxylate esters;

wherein

the carboxyalkyl cellulose ester of component (1) is a C₂-C₄ alkanoate ester of carboxy-C₁-C₃-alkylcellulose having an inherent viscosity of about 0.2 to 0.7 dL/g as measured in a 60/40 by weight solution of phenol/tetrachloroethane at 25° C., a degree of substitution per anhydroglucose unit of carboxy(C₁-C₃-alkyl) of greater than 0.2 to about 0.75, and a degree of substitution per anhydroglucose unit of C₂-C₄ alkanoate ester residue of about 1.5 to 2.7; and

up to about 60 mole percent of the carboxy groups of the carboxyalkyl cellulose ester of component (1) are neutralized with a base.

A preferred embodiment of our invention concerns a nail (finger or toe) coating composition comprising:

(1) about 2 to 25 weight percent, based on the total weight of (1) and (2), of a partially neutralized carboxyalkyl cellulose ester;

(2) about 75 to 98 weight percent, based on the total weight of (1) and (2), of a liquid vehicle comprising

(i) about 10 to 50 weight percent water;

(ii) about 2 to 25 weight percent of an organic solvent that evaporates slower than water selected from ether-alkanols, carboxylate esters of ether-alkanols, mono-carboxylate esters of diols containing from about 6 to 16 carbon atoms, plasticizers, and mixtures of any two or more thereof; and

(iii) about 40 to 88 weight percent of a fast-evaporating solvent selected from one or more aliphatic, straight- or branched-chain alkanols having 2 to 4 carbon atoms, aliphatic, straight-chain ketones having 3 to 5 carbon atoms, and lower alkyl carboxylate esters;

wherein

the carboxyalkyl cellulose ester is a carboxymethyl cellulose propionate, carboxymethyl cellulose acetate propionate, carboxymethyl cellulose butyrate or carboxymethyl cellulose acetate butyrate having a degree of substitution of carboxymethyl of 0.2 to 0.75, a degree of substitution per anhydroglucose unit of hydroxyl from about 0.1 to 0.9, and having an inherent viscosity of 0.2 to 0.7 dL/g, as measured in a 60/40 by weight solution of phenol/tetrachloroethane at 25° C.; wherein the carboxymethyl cellulose propionate has a propionyl DS of about 1.5 to 2.7; the carboxymethyl cellulose acetate propionate has an acetyl DS of about 0.1 to 1.6 and a propionyl DS of about 0.1 to 2.6; the carboxymethyl cellulose butyrate has a butyryl DS of about 1.5 to 2.7; and the carboxymethyl cellulose acetate butyrate has an acetyl DS of about 0.1 to 1.6 and a butyryl DS of about 0.1 to 2.6; and

about 15 to 60 mole percent of the carboxy groups of the carboxyalkyl cellulose ester of component (1) are neutralized with a base. The compositions provided by the present invention utilize a carboxyalkyl cellulose ester as the primary or secondary film-former, reducing the need for odoriferous solvents and providing the opportunity to replace solvent with water and thus reduce VOCs. The carboxyalkyl cellulose esters exhibit solubility in a wide range of organic solvents, compatibility with a variety of co-resins and rheology modifiers, and ease of dispersion in water-borne formulations useful in cosmetic applications. The compositions can be anhydrous or contain up to about 80% water.

A third embodiment of our invention involves the use of the coating compositions described above to produce decorative and/or protective coatings on nails, i.e., human finger and toe nails. This embodiment provides a method for preparing a coating on human finger or toe nails which comprises the steps of:

I. applying a coating of one of the coating compositions defined above to a human finger or toe nail; and

II. allowing the coating to dry.

While primarily useful as nail coatings, the above-described compositions also may be applied to the skin to provide a pigmented coating or glossy finish. Thus a fourth embodiment of our invention concerns a skin coating composition comprising:

(1) about 1 to 10 weight percent, based on the total weight of (1) and (2), of a partially neutralized carboxyalkyl cellulose ester;

(2) about 90 to 99 weight percent, based on the total weight of (1) and (2), of a liquid vehicle comprising:

(i) about 60 to 80 weight percent water;

(ii) about 5 to 20 weight percent of an organic solvent that evaporates slower than water selected from ether-alkanols, carboxylate esters of ether-alkanols, mono-carboxylate esters of diols containing from about 6 to 16 carbon atoms, plasticizers, and mixtures of any two or more thereof; and

(iii) 0 to about 20 weight percent of an alkanol;

wherein

the carboxyalkyl cellulose ester is a carboxymethyl cellulose propionate, carboxymethyl cellulose acetate propionate, carboxymethyl cellulose butyrate or carboxymethyl cellulose acetate butyrate having a degree of substitution of carboxymethyl of 0.2 to 0.75, a degree of substitution per anhydroglucose unit of hydroxyl from about 0.1 to 0.9, and having an inherent viscosity of 0.2 to 0.7 dL/g, as measured in a 60/40 by weight solution of phenol/tetrachloroethane at 25° C.; wherein the carboxymethyl cellulose propionate has a propionyl DS of about 1.5 to 2.7; the carboxymethyl cellulose acetate propionate has an acetyl DS of about 0.1 to 1.6 and a propionyl DS of about 0.1 to 2.6; the carboxymethyl cellulose butyrate has a butyryl DS of about 1.5 to 2.7; and the carboxymethyl cellulose acetate butyrate has an acetyl DS of about 0.1 to 1.6 and a butyryl DS of about 0.1 to 2.6; and

about 15 to 60 mole percent of the carboxy groups of the carboxyalkyl cellulose ester of component (1) are neutralized with a base.

### DETAILED DESCRIPTION OF THE INVENTION

The preparation of the C₂-C₄ alkanoate esters of carboxy(C₁-C₃-alkyl) cellulose component of the novel cosmetic coating compositions of the present invention is described in detail in U.S. Pat. Nos. 5,668,273 and 5,792,856. The carboxyalkyl cellulose esters have a high combined degree of substitution (DS) per anhydroglucose unit on the cellulose backbone of ether and ester functions in the tri-substituted or partially hydrolyzed form, typically less than about 0.90 DS hydroxyl. More specifically, the carboxyalkyl cellulose esters have an inherent viscosity of 0.20 to 0.70 dL/g, preferably 0.30 to 0.70 dL/g, as measured in a 60/40 by weight solution of phenol/tetrachloroethane at 25° C., a degree of substitution per anhydroglucose unit of carboxy(C₁-C₃ alkyl) of about 0.20 to 0.75, and a degree of substitution per anhydroglucose unit of C₂-C₄ alkanoate ester residues, i.e., acetate, propionate and/or butyrate, of about 1.5 to about 2.7, and a degree of substitution per anhydroglucose unit of hydroxyl of about 0.1 to 0.9, preferably 0.1 to 0.7.

The C₂-C₄ alkanoate esters of carboxy(C₁-C₃-alkyl) cellulose component of our novel cosmetic coating compositions preferably is a carboxymethyl cellulose propionate, a carboxymethylcellulose acetate propionate, a carboxymethyl cellulose butyrate, or a carboxymethyl cellulose acetate butyratehaving a degree of substitution of carboxymethyl of 0.2 to 0.75, preferably 0.25 to 0.35, a degree of substitution per anhydroglucose unit of hydroxyl from about 0.1 to 0.7, and a degree of substitution per anhydroglucose unit of acetyl of 0.1 to 1.65, and having an inherent viscosity of 0.2 to 0.7 dL/g, as measured in a 60/40 by weight solution of phenol/tetrachloroethane at 25° C.; wherein the carboxymethyl cellulose propionate has a propionyl DS of about 1.5 to 2.7; the carboxymethyl cellulose acetate propionate has an acetyl DS of about 0.1 to 1.6 and a propionyl DS of about 0.1 to 2.6; the carboxymethyl cellulose butyrate has a butyryl DS of about 1.5 to 2.7; and the carboxymethyl cellulose acetate butyrate has an acetyl DS of about 0.1 to 1.6 and a butyryl DS of about 0.1 to 2.6. The carboxymethyl cellulose acetate butyrates and propionates preferably have an inherent viscosity of 0.35 to 0.60 dL/g, a degree of substitution per anhydroglucose unit of butyryl or propionyl of 1.1 to 2.55, and a degree of substitution per anhydroglucose unit of acetyl is 0.1 to 0.9.

The second component of our novel cosmetic coating compositions is a liquid vehicle comprising (i) 0 to about 80 weight percent water, (ii) about 2 to 25 weight percent of an organic solvent that evaporates slower than water selected from ether-alkanols, carboxylate esters of ether-alkanols, mono-carboxylate esters of diols containing from about 6 to 16 carbon atoms, plasticizers, and mixtures of any two or more thereof; and (iii) 0 to about 88 weight percent of a fast-evaporating solvent selected from one or more aliphatic, straight- or branched-chain alkanols having 2 to 4 carbon atoms; aliphatic, straight-chain ketones having 3 to 5 carbon atoms; and lower alkyl carboxylate esters. The organic solvent portion of the liquid vehicle is comprised of at least one solvent that evaporates slower than water and has a boiling point greater than about 150° C. and, optionally, depending on the desired viscosity and drying rate of the composition, may contain one or more solvents that evaporate faster than water. Both solvent portions are at least partially soluble in water. Examples of organic solvents that evaporate slower than water include ether-alkanols, carboxylate esters of ether-alkanols, mono-carboxylate esters of diols containing from about 6 to 16 carbon atoms and mixtures of any two or more thereof. The slow-evaporating solvent also is referred to as a coalescing aid as its function is to ensure continuous film formation as the composition dries. The slow-evaporating solvent or coalescing aid also can contribute to flow-out, leveling, and gloss. The slow-evaporating solvent may be selected from one or a combination of organic solvents typically used in cosmetic formulations including, for example, 2-butoxy-ethanol, 2-butoxyethyl acetate, 2-(2-ethoxy)ethoxyethanol, 2-(2-ethoxy)ethoxyethyl acetate, 1-propoxy-2-propanol, 2,2,4-trimethylpentane-1,3-diol monoisobutyrate (Texanol ester-alcohol available from Eastman Chemical Company), and 4-hydroxy-4-methyl-2-pentanone (diacetone alcohol). The preferred slow-evaporating solvents are 2-butoxyethanol, 2-(2-ethoxy)ethoxyethanol, and mixtures thereof. The slow-evaporating solvent or coalescing aid typically constitutes about 2 to 25 weight percent, preferably about 3 to 15 weight percent, of the liquid vehicle component of the cosmetic coating compositions. The coalescing aid also may function as a plasticizer, or the coating compositions may contain one or more additional additives that function specifically as a plasticizer. Thus, some or all, preferably from about 30 to 90 weight percent, of the slow evaporating solvent may comprise one or more ester compounds typically used as a plasticizer in polymeric compositions. Such plasticizer ester compounds are described in more detail below.

A fast-evaporating solvent is an optional component of the liquid vehicle depending on the desired viscosity and evaporation rate of the liquid vehicle for the particular cosmetic application. For nail coating compositions, fast or quick drying is desirable and, therefore, nail coating compositions typically contain a fast-evaporating solvent. It is beneficial, although not essential, for the fast-evaporating solvent to form an azeotrope with water to further enhance drying of the composition. The fast-evaporating solvent preferably is an alkanol, ketone, or lower alkyl carboxylate ester, or combinations thereof. The alkanol may be selected from one or more aliphatic, straight- or branched-chain alkanols having 2 to 4 carbon atoms. Ethanol, propanol, isopropanol and mixtures thereof are the preferred alkanols. The ketone may be selected from the aliphatic, straight-chain ketones having 3 to 5 carbon atoms. Acetone and 2-butanone are the preferred ketones. A lower alkyl carboxylate ester, e.g. a lower alkyl acetate ester, preferably ethyl acetate, also may be used in a liquid vehicle. If desired, alkyl carboxylate esters and/or ketones may be omitted from the formulation to reduce odor. Most preferably, the organic, fast-evaporating solvent is ethanol, isopropanol, or mixtures thereof.

The liquid vehicle of the cosmetic coating compositions normally comprises a combination of water and organic solvent. However, nail coating compositions may be anhydrous to provide faster drying. When water is present in the nail coating compositions, the liquid vehicle may contain up to about 50 weight percent water. Preferably, about 10 to 40 weight percent of the liquid vehicle is water and the remainder of the liquid vehicle comprises about 2 to 25 weight percent of an organic solvent that evaporates slower than water, and about 40 to 88 weight percent of an organic solvent that evaporates faster than water.

The cosmetic coating compositions may contain a plasticizer to increase flexibility of the polymer or resin coating or film and improve adhesion to the substrate. Plasticizers also may function as solvents for the film-forming polymer and as coalescing aids. Examples of typical plasticizers used in cosmetic formulations include, but are not limited to, a variety of esters such as triacetin, acetyl tri-C₂-C₆-alkyl citrate, tri-C₂-C₄-alkyl citrate, di-C₁-C₂₀-alkyl adipate, di-C₁-C₄ alkyl phthalate, di-C₁-C₈ alkyl terephthalate, butyl benzyl phthalate, 2,2,4-trimethyl-1,3-pentanediol diisobutyrate, tri-(2-ethylhexyl) trimellitate, sucrose acetate isobutyrate, sucrose benzoate and alkyl phosphates; glycols such as dipropylene glycol, neopentyl glycol, and PEG-800; and camphor. The plasticizers preferred for use in the cosmetic coating compositions are dibutyl phthalate, diethyl phthalate, 2,2,4-trimethyl-1,3-pentanediol diisobutyrate (Eastman TXIB plasticizer available from Eastman Chemical Company), and dipropylene glycol. Most preferred are dibutyl phthalate and diethyl phthalate. When a compound that functions primarily as a plasticizer (rather than as a slow-evaporating solvent or coalescing aid) is included in the cosmetic coating compositions of the present invention, the amount of such plasticizer compound may be in the range of about 10 to 50 weight percent, preferably about 15 to 40 weight percent, based on the weight of the C₂-C₄ alkanoate esters of carboxy(C₁-C₃-alkyl) cellulose, film-forming component or, when more than one film-forming component is present, based on the combined weight of the C₂-C₄ alkanoate esters of carboxy(C₁-C₃-alkyl) cellulose and other film-forming component(s) or ingredients.

Some of the carboxy groups of the C₂-C₄ alkanoate esters of carboxy(C₁-C₃-alkyl) cellulose component of the cosmetic coating composition may be neutralized to provide increased dispersibility or solubility in water-based systems. The degree of neutralization, i.e., the percentage of acid groups that are neutralized with base, is adjusted based on the other ingredients present in the cosmetic coating composition, and the intended function and/or performance characteristics of the cosmetic composition. Compositions containing a coalescing aid form a clear, continuous film without neutralization if the water content is less than about 15 weight percent based on the total weight of the coating composition. For compositions containing a coalescing aid and greater than about 15 weight percent water, the degree of neutralization should be in the range of about 15 to 60%. For greater water resistance, the neutralization should be about 15% to 40%.

The carboxy groups of the C₂-C₄ alkanoate esters of carboxy(C₁-C₃-alkyl) cellulose may be neutralized with a base such as sodium and potassium carbonate, ammonium hydroxide and amines. The base preferably is an amine, preferably an alkanolamine and, most preferably, an alkanolamine having a molecular weight of about 45 to 200. Examples of the alkanolamines include monoethanolamine, diethanolamine, triethanolamine, dimethyl(ethanol)amine, 2-amino-2-methylpropanol (AMP), monoisopropanolamine, triisopropanolamine, and combinations thereof. The preferred neutralizing agent is AMP.

The C₂-C₄ alkanoate esters of carboxy(C₁-C₃-alkyl) cellulose can be used by itself as the only film-former in the composition or in combination with one or more additional film-forming polymers or resins. Other film-formers include, but are not limited to, the secondary resins previously mentioned for solvent-based compositions: acrylate and polyester resins, toluenesulfonamide/epoxy resin, toluenesulfonamide/formaldehyde resin, cellulose acetate propionate, and cellulose acetate butyrate. An example of a polyester resin is Polymac 220-1085 (70% adipic acid/neopentyl glycol/trimellitic anhydride copolymer in butyl acetate) available from Eastman Chemical Company. Film-formers typically used in water-based systems also may be used in combination with the C₂-C₄ alkanoate esters of carboxy(C₁-C₃-alkyl) cellulose; for example, water-dispersible acrylics, water-dispersible polyesters, and combinations thereof. Examples of water-dispersible acrylics include Hydreau Waterborne Polymers IC 800 and IC 801, surfactant-free acrylic styrene emulsions available from Eastman Chemical Company (37% solids in water). The total amount of film-forming resin that can be used in the composition is about 1% to 40%. For a cosmetic nail coating composition the total amount of film former is preferably about 10 to 25%. For a cosmetic composition for the skin, the total amount of film former is preferably about 1 to 10%.

The C₂-C₄ alkanoate esters of carboxy(C₁-C₃-alkyl) cellulose component of our novel cosmetic coating compositions preferably is a carboxymethyl cellulose propionate, carboxymethyl cellulose acetate propionate, carboxymethyl cellulose butyrate or carboxymethyl cellulose acetate butyrate having a degree of substitution of carboxymethyl of 0.2 to 0.75, preferably 0.25 to 0.35, a degree of substitution per anhydroglucose unit of hydroxyl from about 0.1 to 0.9, and a degree of substitution per anhydroglucose unit of butyryl or propionyl of about 0.1 to 2.6 and a degree of substitution per anhydroglucose unit of acetyl of 0.1 to 1.65, and having an inherent viscosity of 0.2 to 0.7 dL/g, as measured in a 60/40 by weight solution of phenol/tetrachloroethane at 25° C. The carboxymethyl cellulose propionate, carboxymethyl cellulose acetate propionate, carboxymethyl cellulose butyrate, and carboxymethyl cellulose acetate butyrate preferably have an inherent viscosity of 0.30 to 0.70 dL/g, a degree of substitution per anhydroglucose unit of hydroxyl of 0.10 to 0.70, a degree of substitution per anhydroglucose unit of butyryl or propionyl of 1.1 to 2.55, and a degree of substitution per anhydroglucose unit of acetyl is 0.1 to 0.9. The preferred carboxymethyl cellulose esters exhibit improved solubility characteristics over carboxymethylcellulose acetate or conventional cellulose esters such as cellulose acetate butyrate or cellulose acetate propionate. These carboxymethyl cellulose esters are soluble in most classes of typical coating solvents including ketones, esters, alcohols, glycol ethers, and glycol ether esters, while tolerating dilution with water.

The performance properties of the cosmetic coating composition can be altered by varying the level of neutralization of the C₂-C₄ alkanoate esters of carboxy(C₁-C₃-alkyl) cellulose component and/or by adjusting the amount and/or type of co-resin, coalescing aid, solvent (including water) or plasticizer. Other conventional cosmetic additives are incorporated to achieve specific functions or effects. The cellulose ester component of the compositions of the present invention are useful as rheology modifiers. Amine-neutralized, waterborne dispersions containing C₂-C₄ alkanoate esters of carboxy(C₁-C₃-alkyl) cellulose at low concentrations, e.g., less than 5 weight percent, exhibit exponential viscosity changes with cellulose ester concentration changes of as little as 0.5 weight percent. This rapid viscosity build is especially useful to keep the composition from running after application to the fingernails or skin. Additionally, the C₂-C₄ alkanoate esters of carboxy(C₁-C₃-alkyl) cellulose are useful as wetting agents, reducing the interfacial tension of the cosmetic coatings of the present invention. This property is useful for the improved wetting and dispersing of pigments, especially metallic flake pigments (see U.S. Pat. No. 5,994,530).

The novel cosmetic coating compositions may contain one or more pigments to color the composition and/or to subsequently color the skin or nails. Also, special-effect pigments such as metallic pigments may be used to achieve effects such as pearlescence or reflectance. The rheology modification and enhanced wetting characteristics mentioned above, provide improved pigmentation of the cosmetic coating compositions of the invention. Depending on the formulation and effect desired, the pigment content of the cosmetic coating composition can range up to about 25 weight percent based on the total weight of the coating composition. Typically, pigments may be incorporated into the cosmetic coating by first preparing a pigment dispersion. The pigments may be dispersed by blending a solution or dispersion of the C₂-C₄ alkanoate esters of carboxy(C₁-C₃-alkyl) cellulose with the pigment using minimal shear. The weight ratio of pigment to cellulose ester in the pigment dispersion may be as high as 90:10. The preferred maximum weight ratio is about 4:1 to 2:1 pigment to CAC ester. Useful pigments are those typically used in cosmetics such as, for example, FD&C Yellow No. 5 Aluminum Lake, D&C Red No. 6 Barium Lake, D&C Red No. 7 Calcium Lake, D&C Violet No. 2, manganese violet, chromium oxide greens, chromium hydroxide green, ferric ammonium ferrocyanide, titanium oxide, iron oxides, bismuth oxychloride and guanine; also metallic pigments such as aluminum, copper, bronze, silver, and mica; and mixtures thereof. The use of the C₂-C₄ alkanoate esters of carboxy(C₁-C₃-alkyl) cellulose provides a stable pigment dispersion, which can easily be incorporated into the cosmetic coating composition. When using metallic flake pigments, the cellulose ester provides excellent flake orientation and good opacity.

The cosmetic coating compositions of this invention may contain other conventional cosmetic additives known in the art in amounts which generally range up to about 20 weight percent, preferably from about 0.01 to about 10 weight percent based on the total weight of the coating composition. Examples of such additives include, but are not limited to, silicones, emollients, emulsifiers, lubricants and skin penetrants, protein hydrolysates, or other protein derivatives, waxes, viscosity increasing and decreasing agents, ethylene adducts and polyoxyethylene cholesterol, dyes, tints, ultraviolet (UV) absorbers, optical brighteners, perfumes or fragrances, antioxidants, preservatives, antifoaming agents, chelating agents, and the like. Examples of these and other types of additives may be found in the International Cosmetic Ingredient Dictionary and Handbook, Eighth Edition, published by the Cosmetic, Toiletry, and Fragrance Association, Inc., 1101 17^{th} Street, N.W., Suite 300, Washington, D.C. 20036-4702. The nail coating compositions may be applied to human finger or toe nails according to conventional techniques using an applicator, typically a brush applicator.

### EXAMPLES

The preparation and use of the cosmetic coating compositions provided by the present invention are further illustrated by the following examples. All percentages given in the examples are by weight unless otherwise specified. The carboxy(C₁-C₃-alkyl) cellulose esters used in the examples were carboxymethyl cellulose acetate butyrate (CMCAB) having an inherent viscosity of 0.40 to 0.41 dL/g, a degree of substitution per anhydroglucose unit of hydroxyl of 0.63 to 0.64, a degree of substitution per anhydroglucose unit of butyryl of 1.63 to 1.66, and a degree of substitution per anhydroglucose unit of acetyl of 0.41 to 0.43.

### Reference Examples 1-10

The liquid masterbatch compositions comprising partially-neutralized CMCAB of Reference Example 1, 3, 5 and 6 set forth in Table I were prepared by combining ethanol and AMP-95 neutralizing agent (95% 2-amino-2-methyl-1-propanol from Angus Chemical Company), slowly adding CMCAB, and mixing until dissolved. Reference Examples 2 and 4 were prepared by slowly adding water to Masterbatch Examples 1 and 3 with rapid stirring. The liquid compositions of Reference Examples 7-10 were prepared by combining the water and alcohol, adding the neutralizing agent AMP-95, adding the CMCAB, and mixing until dissolved. In Table I, the values given for CMCAB, water, ethanol and isopropanol refer to the weight percent present based on the total weight of each composition and Neutral'n refers to the percent of carboxyl groups of the CMCAB neutralized with 2-amino-2-methyl-1-propanol. The values given for each Reference Example do not include a minor amount of AMP-95 neutralizing agent present.

TABLE I

| Reference | | | | | |
|---|---|---|---|---|---|
| Example | CMCAB | Water | Ethanol | Isopropanol | Neutral'n |
| 1 | 30.0 | 0 | 69.4 | 0 | 20 |
| 2 | 17.7 | 41.2 | 40.8 | 0 | 20 |
| 3 | 40.0 | 0 | 59.2 | 0 | 20 |
| 4 | 23.5 | 41.2 | 34.8 | 0 | 20 |
| 5 | 25.0 | 0 | 74.6 | 0 | 15 |
| 6 | 25.0 | 0 | 74.0 | 0 | 40 |
| 7 | 25.0 | 11.2 | 0 | 63.4 | 15 |
| 8 | 25.0 | 11.1 | 0 | 62.9 | 40 |
| 9 | 25.0 | 11.2 | 63.4 | 0 | 15 |
| 10 | 25.0 | 11.1 | 62.9 | 0 | 40 |

the water/ethanol solvent system provided a hazier, higher viscosity CMCAB solution than the water/isopropanol or ethanol without water. Neutralization at 40% resulted in less hazy solutions than 15% neutralization. The neutralization level normally should be sufficient to dissolve all of the CMCAB. The required level will vary depending on the solvent system.

### Examples 1-17

The cosmetic coating compositions of Examples 1-19 were prepared by blending a solvent or plasticizer with one of the liquid masterbatch compositions described above to produce the compositions described in Table II wherein each % value refers to a weight percent based on the total weight of the composition and Reference Example refers to the liquid composition of above-described reference examples used in the preparation of the coating composition.

TABLE II

| Example | Solvent or | | Reference | CMCAB | Ethanol | Water |
|---|---|---|---|---|---|---|
| No. | Plasticizer | % | Example | % | % | % |
| 1 | 2-(2-Butoxyethoxy)ethanol | 8.7 | 4 | 20.5 | 30.3 | 40.2 |
| 2 | 2-Butoxyethanol | 8.7 | 4 | 20.5 | 30.3 | 40.2 |
| 3 | 2-(2-Ethoxyethoxy)ethanol | 9.1 | 2 | 16.1 | 37.1 | 37.5 |
| 4 | 2-(2-Methoxyethoxy)ethanol | 8.7 | 9 | 16.0 | 34.4 | 40.6 |
| 5 | 1-Methoxy-2-Propanol | 8.7 | 4 | 20.5 | 30.3 | 40.2 |
| 6 | Butyl Acetate | 8.7 | 4 | 20.5 | 30.3 | 40.2 |
| 7 | Ethyl Acetate | 8.7 | 4 | 20.5 | 30.3 | 40.2 |
| 8 | 4-Methyl-2-Pentanone | 7.0 | 2 | 16.4 | 38.0 | 38.3 |
| 9 | Dibutyl Phthalate | 9.1 | 2 | 16.1 | 37.1 | 37.5 |
| 10 | Diethyl Phthalate | 7.0 | 2 | 16.4 | 38.0 | 38.3 |
| 11 | Di(2-Ethylhexyl) Adipate | 8.7 | 2 | 20.5 | 30.3 | 40.2 |
| 12 | Dipropylene Glycol | 9.1 | 2 | 16.1 | 37.1 | 37.5 |
| 13 | Eastman SAIB-90EA | 9.1 | 2 | 16.1 | 37.1 | 37.5 |
| 14 | Propylene Glycol | 7.1 | 2 | 16.4 | 38.0 | 38.3 |
| 15 | Triethyl Citrate | 7.1 | 2 | 16.4 | 38.0 | 38.3 |
| 16 | 2,2,4-Trimethyl-1,3- | 9.1 | 2 | 16.1 | 37.1 | 37.5 |
| | Pentanediol Diisobutyrate | | | | | |
| 17 | Tri-(2-Ethylhexyl) Trimellitate | 7.0 | 2 | 16.4 | 38.0 | 38.3 |

Eastman SAIB-90EA consists of 90% sucrose acetate isobutyrate and 10% ethyl acetate.

The coating compositions of Examples 1-17 had a range of viscosities. In general, the compositions containing a slow-evaporating glycol or glycol ether (Examples 1-5, 12 and 14) had a higher viscosity than did the composition containing a fast-evaporating solvent (Examples 6-8). The compositions of Examples 6-11, 13, 15 and 16 had relatively low viscosities whereas the composition of Example 17 had a higher viscosity. Thus, the viscosity of CMCAB-based coating compositions may be adjusted as needed for the specific application through judicious selection of the solvent type and solvent concentration. The plasticizer also may function as a solvent depending on the degree of solubility of CMCAB in the plasticizer. When the slow-evaporating solvent or plasticizer is a good solvent for CMCAB, it functions as a coalescing aid, resulting in a clear, continuous film. All of the compositions were homogeneous and white.

A small amount of the coating composition of Example 2 was spread on the hand. It dried to a glossy film that remained visible after several exposures to soap and water.

Coatings of each of the coating compositions of Examples 1-17 were prepared on glass plates using a 10-mil (254-micron) drawdown cup. The coatings were dried at room temperature and the appearance of each was observed. The water resistance of each coating was determined by observing adhesion and appearance of the coating on the glass plate after exposure to running water. The appearance and water resistance of each coating are reported in Table III.

TABLE III

| Example | | Water |
|---|---|---|
| No. | Appearance | Resistance |
| 1 | Clear, continuous, pitted, adheres | Good |
| 2 | Clear, continuous, adheres | Good |
| 3 | Clear, continuous, adheres | Good |
| 4 | Clear, continuous with crazing, some | Partial |
| | adhesion | |
| 5 | Flaky, no adhesion | None |
| 6 | Powdery, flaky, some adhesion | Partial |
| 7 | Flaky, some adhesion | Partial |
| 8 | Hazy, flaky, no adhesion | None |
| 9 | Clear, continuous with slight orange peel | Good |
| 10 | Clear, continuous with slight orange peel | Good |
| 11 | Dull, opaque, powdery, no adhesion | None |
| 12 | Clear, continuous with slight crazing | Good |
| 13 | Flaky, no adhesion | None |
| 14 | Clear, scaly, some adhesion | Partial |
| 15 | Clear, irregular with substantial orange peel | None |
| 16 | Clear, uneven with crazing | Good |
| 17 | White, powdery, no adhesion | None |

The coating compositions which produce coatings having low or no water resistance may be used as skin coatings wherein water removability may be desirable.

### Examples 18-25

Examples 18-25 show the effect of solvents and plasticizers in combination with CMCAB as the primary resin and Polymac 220-1085 polyester as the secondary resin. Polymac 220-1085 polyester resin consists of 70 weight percent of a copolyester prepared from adipic acid, neopentyl glycol and trimellitic anhydride in butyl actetate and is available from Eastman Chemical Company. The coating compositions of Examples 18-25 were prepared by combining one or more solvents with liquid masterbatch of Reference Example 2 and then adding a plasticizer and the Polymac 220-1085 polyester co-resin. The compositions were mixed after each addition. The compositions Examples 18-25 are described in Table IV wherein each % value refers to a weight percent based on the total weight of the composition and Masterbatch refers to the liquid masterbatch composition of Reference Example 2.

TABLE IV

| Example | Masterbatch | Polymac | | | | |
|---|---|---|---|---|---|---|
| No. | % | % | Solvent | % | Plasticizer | % |
| 18 | 90.9 | 6.8 | 2-Butoxyethanol | 2.3 | None | 0 |
| 19 | 92.5 | 1.7 | 2-Butoxyethanol | 0.6 | Diethyl Phthalate | 5.2 |
| 20 | 90.8 | 1.7 | 2-Butoxyethanol | 0.6 | None | 0 |
| | | | 2-(Ethoxyethoxy)ethanol | 6.9 | | |
| 21 | 90.9 | 1.4 | 2-Butoxyethanol | 0.5 | Dibutyl Phthalate | 6.2 |
| | | | 2-(Ethoxyethoxy)ethanol | 1.0 | | |
| 22 | 92.8 | 2.0 | 2-(Ethoxyethoxy)ethanol | 1.2 | Dipropylene Glycol | 4.0 |
| 23 | 92.8 | 2.0 | 2-Butoxyethanol | 1.2 | Dipropylene Glycol | 4.0 |
| 24 | 92.8 | 2.0 | 2-(Ethoxyethoxy)ethanol | 1.2 | Dibutyl Phthalate | 4.0 |
| 25 | 92.8 | 2.0 | 2-Butoxyethanol | 1.2 | Dibutyl Phthalate | 4.0 |

The coating compositions of Examples 18-25 were white and homogeneous and varied in viscosity from relatively high (Examples 18, 22, and 23) to medium (Example 20) to low (Examples 19, 21, 24 and 25). Examples 22-25 compare dibutyl phthalate and dipropylene glycol plasticizers. Dibutyl phthalate provided lower-viscosity compositions and coatings having better water resistance as compared to dipropylene glycol.

Coatings of each of the coating compositions of Examples 18-25 were prepared on glass plates using a 10-mil (254-micron) drawdown cup. The coatings were dried at room temperature and the appearance of each was observed. The water resistance of each coating was determined by observing adhesion and appearance of the coating on the glass plate after exposure to running water. The appearance and water resistance of each coating are reported in Table V.

TABLE V

| Example | | |
|---|---|---|
| No. | Appearance | Water Resistance |
| 18 | Clear, uneven, adheres | Good |
| 19 | Clear, continuous, glossy, adheres | Good |
| 20 | Clear, continuous, slight orange peel | Partial |
| | adheres | |
| 21 | Clear, continuous, slight orange peel, | Good |
| | glossy, adheres | |
| 22 | Clear, flaky, no adhesion | None |
| 23 | Clear, uneven, adheres | None |
| 24 | Clear, continuous, slight orange peel, | Good |
| | adheres | |
| 25 | Clear, continuous, slight orange peel, | Good |
| | adheres | |

### Examples 26-37

The coating compositions of Examples 26-37 were prepared by combining 2-butoxyethanol, dibutyl phthalate, and, optionally, Polymac 220-1085 resin with one of the liquid masterbatch compositions of Reference Examples 5-10. Each of the coating compositions contained 1 weight percent 2-butoxyethanol, 6.2 weight percent dibutyl phthalate and 64 weight percent of one of the liquid masterbatch compositions. Additional ethanol was added to the anhydrous formulations (Examples 26-29) and additional water was added to the aqueous formulations (Examples 30-37). In the compositions without Polymac polyester resin, additional ethanol (EtOH) or water is added in place of the Polymac resin. Examples 26-37 compare CMCAB coating compositions wherein 15% or 40% of the carboxy groups of the CMCAB are neutralized without or with Polymac 220-1085 polyester resin as the secondary resin. The compositions of Examples 26-37 are described in Table VI wherein Masterbatch R.E. refers to the liquid masterbatch of the specified Reference Example (R.E.) used in each coating composition, % N refers to the percentage carboxy groups of the CMCAB that are neutralized, EtOH refers to ethanol and the other % values refer to a weight percent based on the total weight of the composition.

TABLE VI

| Example | Masterbatch | | Polymac | Additional | |
|---|---|---|---|---|---|
| No. | R.E. | % N | % | EtOH or Water | % |
| 26 | 5 | 15 | 0 | Ethanol | 28.8 |
| 27 | 5 | 15 | 1.5 | Ethanol | 27.3 |
| 28 | 6 | 40 | 0 | Ethanol | 28.8 |
| 29 | 6 | 40 | 1.5 | Ethanol | 27.3 |
| 30 | 7 | 15 | 0 | Water | 28.8 |
| 31 | 7 | 15 | 1.5 | Water | 27.3 |
| 32 | 8 | 40 | 0 | Water | 28.8 |
| 33 | 8 | 40 | 1.5 | Water | 27.3 |
| 34 | 9 | 15 | 0 | Water | 28.8 |
| 35 | 9 | 15 | 1.5 | Water | 27.3 |
| 36 | 10 | 40 | 0 | Water | 28.8 |
| 37 | 10 | 40 | 1.6 | Water | 27.3 |

The coating compositions of Examples 26-37 were homogeneous and varied in viscosity from medium (Examples 30-33, 36, and 37) to low (Examples 26-29, 34, and 35). The aqueous coating compositions containing 40% neutralized CMCAB were less opaque than those containing 15% neutralized CMCAB. Otherwise, the level of neutralization had little effect on the formulation or coating properties. In all of the examples, Polymac 220-1085 polyester co-resin increased the hardness of the film compared to using CMCAB without the co-resin.

Coatings of each of the coating compositions of Examples 26-37 were prepared on glass plates using a 10-mil (254-micron) drawdown cup. The coatings were dried at room temperature and the appearance of each was observed. The water resistance of each coating was determined by observing adhesion and appearance of the coating on the glass plate after exposure to running water. The appearance and water resistance of each coating are reported in Table VII.

TABLE VII

| Example | | |
|---|---|---|
| No. | Appearance | Water Resistance |
| 26 | Clear, orange peel, continuous, adheres | Good |
| 27 | Clear, orange peel, continuous, adheres | Good |
| 28 | Clear, orange peel, continuous, adheres | Good |
| 29 | Clear, orange peel, continuous, adheres | Partial |
| 30 | Clear, continuous, uneven edges, adheres | Good |
| 31 | Clear, continuous, uneven, adheres | Good |
| 32 | Clear, continuous, scaly, adheres | Good |
| 33 | Clear, continuous, scaly, adheres | Good |
| 34 | Clear, continuous, adheres | Good |
| 35 | Clear, continuous, uneven, adheres | Good |
| 36 | Clear, continuous, adheres | Good |
| 37 | Clear, continuous, a few air bubbles, | Good |
| | adheres | |

### Examples 38-48

Examples 38-48 illustrate coating compositions containing a partially-neutralized CMCAB and a waterborne acrylic/styrene polymer as film-forming materials. The waterborne acrylic/styrene polymers employed were Hydreau IC 800 and IC 801 latex polymers, 37 weight percent solids in water, available from Eastman Chemical company. The cosmetic coating compositions of Examples 38-48 were prepared by diluting the Hydreau waterborne acrylic/styrene emulsion(s) with additional water, then mixing with one of the CMCAB masterbatch compositions of Reference Examples 7-10. A mixture of 2-butoxyethanol (Butoxyethanol), dibutyl phthalate and, optionally, 2-(2-ethoxyethoxy)ethanol (Ethoxydiglycol) was added last. The CMCAB used in the compositions of Examples 38-46 was 40% neutralized whereas the CMCAB used in the compositions of Examples 47 and 48 was 15% neutralized. The cosmetic coating compositions of Examples 38-48 are described in detail in Table VIII wherein Masterbatch R.E. refers to the liquid masterbatch of the specified Reference Example (R.E.) used in each coating composition and the % values refer to a weight percent based on the total weight of the composition.

TABLE VIII

| Example | Masterbatch | | Hydreau | | Solvent | | Dibutyl | Coating Composition |
|---|---|---|---|---|---|---|---|---|
| No. | R.E. | % | Polymer | % | | % | Phthalate, % | Characteristics |
| 38 | 8 | 64.0 | IC 800 | 6.0 | Water | 24.0 | 5.2 | Off-white, medium |
| | | | | | Butoxyethanol | 0.8 | | viscosity, homogeneous |
| 39 | 8 | 64.0 | IC 801 | 6.0 | Water | 24.0 | 5.2 | Off-white, medium |
| | | | | | Butoxyethanol | 0.8 | | viscosity, homogeneous |
| 40 | 8 | 64.0 | IC 800 | 4.0 | Water | 24.0 | 5.2 | Off-white, medium |
| | | | IC 801 | 2.0 | Butoxyethanol | 0.8 | | viscosity, homogeneous |
| 41 | 8 | 63.4 | IC 800 | 5.9 | Water | 23.8 | 5.1 | Off-white, medium |
| | | | | | Butoxyethanol | 0.8 | | viscosity, homogeneous |
| | | | | | Ethoxydiglycol | 1.0 | | |
| 42 | 8 | 63.4 | IC 801 | 5.9 | Water | 23.8 | 5.1 | Off-white, medium |
| | | | | | Butoxyethanol | 0.8 | | viscosity, homogeneous |
| | | | | | Ethoxydiglycol | 1.0 | | |
| 43 | 8 | 63.4 | IC 800 | 3.9 | Water | 23.8 | 5.1 | Off-white, medium |
| | | | IC 801 | 2.0 | Butoxyethanol | 0.8 | | viscosity, homogeneous |
| | | | | | Ethoxydiglycol | 1.0 | | |
| 44 | 10 | 64.0 | IC 800 | 6.0 | Water | 24.0 | 5.2 | Off-white, high viscosity, |
| | | | | | Butoxyethanol | 0.8 | | homogeneous |
| 45 | 10 | 64.0 | IC 801 | 6.0 | Water | 24.0 | 5.2 | Off-white, high viscosity, |
| | | | | | Butoxyethanol | 0.8 | | homogeneous |
| 46 | 10 | 64.0 | IC 800 | 4.0 | Water | 24.0 | 5.2 | Off-white, high viscosity, |
| | | | IC 801 | 2.0 | Butoxyethanol | 0.8 | | homogeneous |
| 47 | 7 | 64.0 | IC 800 | 5.0 | Water | 24.0 | 5.2 | White, medium viscosity, |
| | | | IC 801 | 1.0 | Butoxyethanol | 0.8 | | homogeneous |
| 48 | 9 | 64.0 | IC 800 | 4.0 | Water | 24.0 | 5.2 | White, medium viscosity, |
| | | | IC 801 | 2.0 | Butoxyethanol | 0.8 | | homogeneous |

Coatings of each of the coating compositions of Examples 38-48 were prepared on glass plates using a 10-mil (254-micron) drawdown cup. The coatings were dried at room temperature and the appearance of each was observed. The water resistance of each coating was determined by observing adhesion and appearance of the coating on the glass plate after exposure to running water. The appearance and water resistance of each coating are reported in Table IX.

TABLE IX

| Example | | |
|---|---|---|
| No. | Appearance | Water Resistance |
| 38 | Clear, continuous, adheres | Partial |
| 39 | Clear, continuous, adheres | Partial |
| 40 | Clear, continuous, adheres | Partial |
| 41 | Clear, continuous, adheres | Good |
| 42 | Clear, continuous, adheres | Good |
| 43 | Clear, continuous, adheres | Partial |
| 44 | Slightly hazy, continuous, adheres | Partial |
| 45 | Slightly hazy, continuous, adheres | Partial |
| 46 | Slightly hazy, continuous, adheres | Partial |
| 47 | Clear, continuous, adheres | Good |
| 48 | Slightly hazy with crazing, continuous, | Partial |
| | adheres | |

The coating compositions containing both butoxyethanol and ethoxydiglycol produced coatings with better water resistance than those containing only butoxyethanol. The presence of Hydreau IC 800 waterborne polymer provided coatings having better gloss compared to coatings prepared from compositions containing Hydreau IC 801. When applied to the fingernails, the coating composition of Example 41 provided a durable and glossy coating.

### Example 49

An anhydrous cosmetic coating composition was prepared from the following components by mixing the components in the order listed:

| | |
|---|---|
| Reference Example 5 CMCAB Masterbatch | 64.0% |
| Amphomer 28-4910 Mixture (16.7% in | 18.0% |
| Ethanol) | |
| 2-Butoxyethanol | 0.8% |
| Dibutyl phthalate | 5.2% |

The Amphomer 28-4910 (an octylacrylamide/acrylates/butylaminoethyl methacrylate copolymer) available from National Starch was neutralized 60% with AMP. Amphomer is an amphoteric acrylic polymer with an acidity of 2.05 me/g. The resulting light-amber composition was homogeneous and had medium viscosity. On a glass plate, it produced a clear, continuous film with slight orange peel and exhibited good adhesion to glass. It gave a glossy coating when applied on the fingernails.

### Example 50

A pigmented hydroalcoholic concentrate was prepared by combining 97.1% Masterbatch Reference Example 2 with 2.9% D&C Red #7 Calcium Lake pigment and mixing until homogeneous. The pigmented concentrate (6.3%) then was combined with 93.7% of the composition of Example 25 and mixed until homogeneous. The resulting mixture was dark pink with low viscosity. It produced a continuous film on a glass plate. When subjected to the water test, the film adhered to the glass.

### Example 51

An anhydrous cosmetic coating composition was prepared from the following components by mixing the components in the order listed

| | |
|---|---|
| 2-Butoxyethanol | 2.0% |
| 2,2,4-Trimethylpentane-1,3-diol Diisobutyrate | 2.0% |
| Dibutyl phthlate | 2.0% |
| Reference Example 1 CMCAB Masterbatch | 91.1% |
| Polymac 220-1085 polyester | 2.9% |

The resulting homogeneous mixture was amber and viscous and produced a continuous, clear film on a glass plate. When subjected to the water test, the film adhered to the glass.

### Example 52

A pigmented anhydrous concentrate was prepared by combining 97.1% Masterbatch Reference Example 1 with 2.9% D&C Red #7 Calcium Lake pigment and mixing until homogeneous. The pigmented concentrate (6.3%) was combined with 93.7% of the composition of Example 51 and mixed until homogeneous. The resulting mixture was red and viscous and produced a continuous film on a glass plate. When subjected to the water test, the film adhered to the glass.

The invention has been described in detail with particular reference to preferred embodiments thereof, but it will be understood that variations and modifications can be effected within the spirit and scope of the invention.

We claim:

1. A cosmetic coating composition comprising:
(1) about 1 to 40 weight percent, based on the total weight of components (1) and (2), of an unneutralized or partially neutralized carboxyalkyl cellulose ester; and
(2) about 60 to 99 weight percent, based on the total weight of components (1) and (2), of a liquid vehicle comprising:
(i) 0 to about 80 weight percent water;
(ii) about 2 to 25 weight percent of an organic solvent that evaporates slower than water selected from ether-alkanols, carboxylate esters of ether-alkanols, mono-carboxylate esters of diols containing from about 6 to 16 carbon atoms, plasticizers, and mixtures of any two or more thereof; and
(iii) 0 to about 88 weight percent of a fast-evaporating solvent selected from one or more aliphatic, straight- or branched-chain alkanols having 2 to 4 carbon atoms, aliphatic, straight-chain ketones having 3 to 5 carbon atoms, and lower alkyl carboxylate esters;
wherein
the carboxyalkyl cellulose ester of component (1) is a C₂-C₄ alkanoate ester of carboxy-C₁-C₃-alkylcellulose having an inherent viscosity of about 0.2 to 0.7 dL/g as measured in a 60/40 by weight solution of phenol/tetrachloroethane at 25° C., a degree of substitution per anhydroglucose unit of carboxy(C₁-C₃-alkyl) of greater than 0.2 to about 0.75, and a degree of substitution per anhydroglucose unit of C₂-C₄ alkanoate ester residue of about 1.5 to 2.7; and
up to about 60 mole percent of the carboxy groups of the carboxyalkyl cellulose ester of component (1) are neutralized with a base.

2. A cosmetic coating composition according to claim 1 wherein the carboxyalkyl cellulose ester has an inherent viscosity of about 0.3 to 0.7 dL/g; a degree of substitution per anhydroglucose unit of hydroxyl of about 0.1 to 0.9; and the liquid vehicle comprises about 3 to 15 weight percent of the organic solvent that evaporates slower than water.

3. A cosmetic coating composition according to claim 2 wherein the the organic solvent that evaporates slower than water comprises about 30 to 90 weight percent of a plasticizer and the carboxyalkyl cellulose ester is a carboxymethyl cellulose ester partially neutralized with an alkanolamine having a molecular weight of about 45 to 200.

4. A finger or toe nail coating composition comprising:
(1) about 2 to 25 weight percent, based on the total weight of components (1) and (2), of a partially neutralized carboxyalkyl cellulose ester;
(2) about 75 to 98 weight percent, based on the total weight of components (1) and (2), of a liquid vehicle comprising:
(i) about 10 to 50 weight percent water;
(ii) about 2 to 25 weight percent of an organic solvent that evaporates slower than water selected from ether-alkanols, carboxylate esters of ether-alkanols, mono-carbxylate esters of diols containing from about 6 to 16 carbon atoms, plasticizers, and mixtures of any two or more thereof; and
(iii) about 40 to 88 weight percent of a fast-evaporating solvent selected from one or more aliphatic, straight- or branched-chain alkanols having 2 to 4 carbon atoms, aliphatic, straight-chain ketones having 3 to 5 carbon atoms, and lower alkyl carboxylate esters;
wherein
the carboxyalkyl cellulose ester is a carboxymethyl cellulose propionate, carboxymethyl cellulose acetate propionate, carboxymethyl cellulose butyrate or carboxymethyl cellulose acetate butyrate having a degree of substitution of carboxymethyl of 0.2 to 0.75, a degree of substitution per anhydroglucose unit of hydroxyl from about 0.1 to 0.9, and having an inherent viscosity of 0.2 to 0.7 dL/g, as measured in a 60/40 by weight solution of phenol/tetrachloroethane at 25° C.; wherein the carboxymethyl cellulose propionate has a propionyl DS of about 1.5 to 2.7; the carboxymethyl cellulose acetate propionate has an acetyl DS of about 0.1 to 1.6 and a propionyl DS of about 0.1 to 2.6; the carboxymethyl cellulose butyrate has a butyryl DS of about 1.5 to 2.7; and the carboxymethyl cellulose acetate butyrate has an acetyl DS of about 0.1 to 1.6 and a butyryl DS of about 0.1 to 2.6; and
about 15 to 60 mole percent of the carboxy groups of the carboxyalkyl cellulose ester of component (1) are neutralized with a base.

5. A finger or toe nail coating composition according to claim 4 wherein the organic solvent that evaporates slower than water is selected from 2-butoxyethanol, 2-(2-ethoxy)ethoxyethanol and mixtures thereof; the composition contains about 30 to 90 weight percent, based on the total weight of the organic solvent that evaporates slower than water, of a plasticizer selected from diethyl phthalate, dibutyl phthalate, and mixtures thereof; and the carboxyalkyl cellulose ester is partially neutralized with an alkanolamine having a molecular weight of about 45 to 200.

6. A finger or toe nail coating composition according to claim 5 wherein the fast-evaporating solvent is selected from ethanol, isopropanol, or mixtures thereof.

7. A finger or toe nail coating composition according to claim 5 wherein the carboxyalkyl cellulose ester is partially neutralized with 2-amino-2-methylpropanol and the fast-evaporating solvent is selected from ethanol, isopropanol, or mixtures thereof.

8. A method for preparing a decorative or protective coating on human toe or finger nails which comprises the steps of:
I. appying a coating of the coating composition defined in claim 1 to a human finger or toe nail; and
II. allowing the coating to dry.

9. A method for preparing a decorative or protective coating on human toe or finger nails which comprises the steps of:
I. appying a coating of the coating composition defined in claim 4 to a human finger or toe nail; and
II. allowing the coating to dry.

10. A method for preparing a decorative or protective coating on human toe or finger nails which comprises the steps of:
I. appying a coating of the coating composition defined in claim 7 to a human finger or toe nail; and
II. allowing the coating to dry.

11. A skin coating composition comprising:
(1) about 1 to 10 weight percent, based on the total weight of components (1) and (2), of a partially neutralized carboxyalkyl cellulose ester;
(2) about 90 to 99 weight percent, based on the total weight of components (1) and (2), of a liquid vehicle comprising:
(i) about 60 to 80 weight percent water;
(ii) about 5 to 20 weight percent of an organic solvent that evaporates slower than water selected from ether-alkanols, carboxylate esters of ether-alkanols, mono-carboxylate esters of diols containing from about 6 to 16 carbon atoms, plasticizers, and mixtures of any two or more thereof; and
(iii) 0 to about 20 weight percent of an alkanol
wherein
the carboxyalkyl cellulose ester is a carboxymethyl cellulose propionate, carboxymethyl cellulose acetate propionate, carboxymethyl cellulose butyrate or carboxymethyl cellulose acetate butyrate having a degree of substitution of carboxymethyl of 0.2 to 0.75, a degree of substitution per anhydroglucose unit of hydroxyl from about 0.1 to 0.9, a degree of substitution per anhydroglucose unit of butyryl or propionyl of about 0.1 to 2.6 and a degree of substitution per anhydroglucose unit of acetyl of 0.1 to 1.65, and having an inherent viscosity of 0.2 to 0.7 dL/g, as measured in a 60/40 by weight solution of phenol/tetrachloroethane at 25° C.; wherein the carboxymethyl cellulose propionate has a propionyl DS of about 1.5 to 2.7; the carboxymethyl cellulose acetate propionate has an acetyl DS of about 0.1 to 1.6 and a propionyl DS of about 0.1 to 2.6; the carboxymethyl cellulose butyrate has a butyryl DS of about 1.5 to 2.7; and the carboxymethyl cellulose acetate butyrate has an acetyl DS of about 0.1 to 1.6 and a butyryl DS of about 0.1 to 2.6; and
about 15 to 60 mole percent of the carboxy groups of the carboxyalkyl cellulose ester of component (1) are neutralized with a base.
